(19) United States
(12) Patent Application Publication
(10) Pub. No.: US 2008/0269891 A1
(43) Pub. Date: Oct. 30, 2008

(54) INTRAOCULAR LENS WITH EDGE MODIFICATION

(75) Inventors: Xin Hong, Arlington, TX (US); Xiaoxiao Zhang, Fort Worth, TX (US)

Correspondence Address: ALCON, IP LEGAL, TB4-8, 6201 SOUTH FREEWAY, FORT WORTH, TX, 76134, US

(73) Assignee: ALCON, INC., Fort Worth, TX (US)

(21) Appl. No.: 11/742,202

(22) Filed: Apr. 30, 2007

(51) Int. Cl. A61F 2/16

(52) U.S. Cl. 623/6.46

(57) ABSTRACT

Intraocular lenses (IOLs) with modified edge characteristics are disclosed to inhibit transverse propagation of internally reflected light rays in order to alleviate, and preferably eliminate, dysphotopsia and/or the perception of dark shadows reported by some users. In one embodiment, IOL designs are disclosed that incorporate an opaque edge or other mechanisms for capturing internally reflected peripheral light rays. In other embodiments, the peripheral region can include a light scattering material or can have a disproportional thickness or be contoured to redirect peripheral rays.

### RELATED APPLICATIONS

This application is related to the following patent applications that are concurrently filed herewith: “Intraocular Lens with Asymmetric Optics,” (Attorney Docket No. 3360); “Intraocular Lens with Peripheral Region Designed to Reduce Negative Dysphotopsia” (Attorney Docket No. 2817); “IOL Peripheral Surface Designs To Reduce Negative Dysphotopsia” (Attorney Docket No. 3345); “Intraocular Lens with Asymmetric Haptics” (Attorney Docket No. 3227); “A New Ocular Implant to Correct Dysphotopsia, Glare, Halo, and Dark Shadow,” (Attorney Docket No. 3226); “Graduated Blue-Filtering Intraocular Lens” (Attorney Docket 2962) and “Haptic Junction Designs to Reduce Negative Dysphotopsia.” (Attorney Docket No. 3344), each of which is incorporated herein by reference.

### BACKGROUND

The present invention relates generally to intraocular lenses (IOLs), and particularly to IOLs that provide a patient with an image of a field of view without the perception of visual artifacts in the peripheral visual field.

The optical power of the eye is determined by the optical power of the cornea and that of the natural crystalline lens, with the lens providing about a third of the eye's total optical power. The process of aging as well as certain diseases, such as diabetes, can cause clouding of the natural lens, a condition commonly known as cataract, which can adversely affect a patient's vision.

Intraocular lenses are routinely employed to replace such a clouded natural lens. Although such IOLs can substantially restore the quality of a patient's vision, some patients with implanted IOLs report aberrant optical phenomena, such as halos, glare or dark regions in their vision. These aberrations are often referred to as “dsyphotopsia.” In particular, some patients report the perception of dark shadows, particularly in their temporal peripheral visual fields. This phenomenon is generally referred to as “negative dsyphotopsia.”

Accordingly, there is a need for enhanced IOLs, especially IOLs that can reduce dysphotopsia, in general, and the perception of dark shadows or negative dysphotopsia, in particular.

### SUMMARY OF THE INVENTION

Intraocular lenses (IOLs) with modified edge characteristics are disclosed to inhibit transverse propagation of internally reflected light rays in order to alleviate, and preferably eliminate, dysphotopsia and/or the perception of dark shadows reported by some users. In one embodiment, IOL designs are disclosed that incorporate an opaque edge or other mechanisms for capturing internally reflected peripheral light rays. In other embodiments, the peripheral region can include a light scattering material or can have a disproportional thickness or be contoured to redirect peripheral rays.

The present invention is based, in part, on the discovery that the shadows perceived by IOL patients can be caused by a double imaging effect when light enters the eye at very large visual angles. More specifically, it has been discovered that in many conventional IOLs, most of the light entering the eye is focused by the cornea and the IOL onto the retina, but some of the light is internally reflected and misdirected by the IOL. This leads to the formation of a second peripheral image offset from the principal image. Either the image itself or the perception of a shadow can be distracting for some lens users.

To reduce the potential complications of cataract surgery, designers of modern IOLs have sought to make the optical component (the “optic”) smaller (and preferably foldable) so that it can be inserted into the capsular bag with greater ease following the removal of the patient's nature crystalline lens. The reduced lens diameter, and foldable lens materials, are important factors in the success of modern IOL surgery, since they reduce the size of the corneal incision that is required. This in turn results in a reduction in corneal aberrations from the surgical incision, and since often no suturing is required. The use of self-sealing incisions results in rapid rehabilitation and further reductions in induced aberrations.

However, to achieve a small optic size, it is typically necessary to use materials with a higher index of refraction. One consequence of the higher index of refraction is greater amount of peripheral light rays entering the eye at high incident angles do not pass through the lens but instead are internally reflected.

Moreover, the use of enhanced polymeric materials and other advances in IOL technology have led to a substantial reduction in capsular opacification, which has historically occurred after the implantation of an IOL in the eye, e.g., due to cell growth. Surgical techniques have also improved along with the lens designs, and biological material that previously could affect light near the edge of an IOL, and in the region surrounding the IOL, no longer does so. These improvements have resulted in a better peripheral vision, as well as a better foveal vision, for the IOL users. Though a peripheral image is not seen as sharply as a central (axial) image, peripheral vision can be very valuable. For example, peripheral vision can alert IOL users to the presence of an object in their field of view, in response to which they can turn to obtain a sharper image of the object. In some IOL users, however, the reduction in capsular opacification can lead to, or exacerbate, sidewise dispersion of internally reflected light and the perception of peripheral visual artifacts, such as dysphotopsia (and/or negative dysphotopsia).

In one aspect of the invention, intraocular lens (IOL) designs are disclosed that incorporate mechanisms for reducing total internal reflection of peripheral light rays. For example, the IOLs of the present invention can include an optic with at least one peripheral region adapted to inhibit transverse propagation of internally reflected light rays. The peripheral region can incorporate a light absorbing material or can comprise an opaque coating on at least a portion of a peripheral edge of the optic, e.g., on at least a portion of the edge, a posterior surface or anterior surface of the peripheral region of the optic or on any combination of these surfaces.

Alternatively, the IOL can further comprise a light scattering composition on or within at least a portion of the peripheral region of the optic, e.g., by incorporation of scattering particles into the body of the optic or via a light scattering composition that is a coating on at least a portion of the peripheral region of the optic.

In another embodiment, the IOL can further comprise a curved surface on at least a portion of the peripheral region of the optic or a tapered edge on at least a portion of the peripheral region of the optic. The curved or tapered edge can further comprise an opaque region or light scattering region. The peripheral region can further comprise at least one refractive surface adapted to redirect said peripheral light rays.

In another aspect of the invention, methods of treatment are disclosed, whereby an eye surgeon can assess the need for iris alignment (or decentration) and then select an IOL having a desired degree of peripheral modification or opacification for implantation. The IOL is preferably folded and inserted into the eye in the folded state. Following passage through the sclera and into the capsular bag, the IOL is unfolded and rotated to the desired orientation to ensure alignment with the center of the iris.

Accordingly, a method of reducing visual artifacts in an eye with an implanted intraocular lens (IOL) is disclosed, in which an IOL is provided having an optic having an opaque edge or other mechanism for reducing total internal reflection of peripheral light rays (or redirecting such rays), and the IOL is implanted into an eye of a patient.

In yet another aspect of the invention, methods of manufacture are disclosed whereby an optic with edge modifications is formed. The method of manufacturing can also include the steps of forming a first haptic, forming a second haptic, and joining the first and second haptics to the edge-modified optic such that the assembly is adapted for use as an intraocular lens. The step of forming and joining can be done sequentially or they can be simultaneous, especially when the haptics and optic are made from the same material.

Further understanding of various aspects of the invention can be obtained by reference to the following detailed description in conjunction with the associated drawings, which are described briefly below.

### BRIEF DESCRIPTION OF THE DRAWINGS

FIG. 1 is a schematic illustration of the problem of internal reflection of peripheral light rays by a conventional intraocular lens (IOL),

FIG. 2 is a schematic top view of an intraocular lens (IOL) with an internal reflection reducing peripheral region according to the invention,

FIG. 3 is a schematic cross-sectional side view of one embodiment of an IOL according to the invention,

FIG. 4 is a schematic cross-sectional side view of another embodiment of an IOL according to the invention,

FIG. 5 is a schematic cross-sectional side view of another embodiment of an IOL according to the invention,

FIG. 6 is a schematic cross-sectional side view of another embodiment of an IOL according to the invention,

FIG. 7 is a schematic cross-sectional side view of another embodiment of an IOL according to the invention,

FIG. 8 is a schematic cross-sectional side view of another embodiment of an IOL according to the invention,

FIG. 9 is a schematic cross-sectional side view of another embodiment of an IOL according to the invention, and

FIG. 10 is a schematic cross-sectional side view of another embodiment of an IOL according to the invention.

### DETAILED DESCRIPTION

The term “intraocular lens” and its abbreviation “IOL” are used herein interchangeably to describe lenses that are implanted into the interior of the eye to either replace the eye's natural lens or to otherwise augment vision regardless of whether or not the natural lens is removed. Phakic lenses, for example, are examples of lenses that may be implanted into the eye without removal of the natural lens.

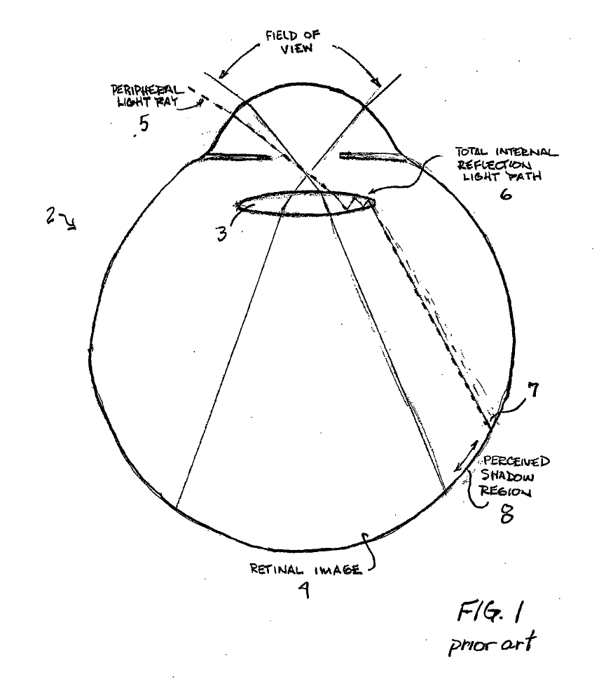

To illustrate the problem of internal reflection-induced dysphotopsia, FIG. 1 shows a conventional IOL 3 implanted in an eye 2. The conventional IOL 3 will form an image 4 of a field of view by focusing a plurality of light rays entering the eye onto the retina. Peripheral light rays (such as ray 5) that enter the eye at large visual angles enter the IOL 3, but can be subject to internal reflection rather than pass through the IOL 3 and form part of the retinal image 4. These high-angle, peripheral rays instead follow an internal reflection path 6 and may reach the retina at a location separated from the image 4 to form either a secondary image or other visual artifact 7. The misdirected light can also result in the perception of a shadow-like phenomenon 8 (negative dysphotopsia) between those images by the patient. Other factors that contribute to these phenomena include depth of IOL implantation (the distance between the IOL and the iris) and the patient's mean pupil size.

FIG. 2 shows an intraocular lens (IOL) 10 having an optic 12, a first haptic 14 and a second haptic 16. Optic 12 further includes a peripheral region 18 adapted to inhibit transverse propagation of internally reflected light rays. (Although peripheral region 18 of FIG. 2 is shown as a radially symmetric region that encircles the entire outer edge of the optic 12, it should be clear that non-symmetric and/or partially encircling peripheral regions are also contemplated and encompassed by the term “peripheral region.”)

The optic and haptics described above can be made as separate pieces attached together or as one-piece of a polymeric material such as acrlyates, e.g., polymethylmethacrylates (PMMAs), or polypropylenes or other foldable materials such as silicones, hydrogels or acrylics. It is usually desirable that the IOL be foldable for insertion to the eye through a small incision and then unfolded when positioned in the eye.

The optic is preferably formed of a biocompatible material, such as soft acrylic, silicone, hydrogel, or other biocompatible polymeric materials having a requisite index of refraction for a particular application. For example, in some embodiments, the optic can be formed of a cross-linked copolymer of 2-phenylethyl acrylate and 2-phenylethyl methacrylate, which is commonly known as Acrysof®.

Generally speaking, the haptics described above serve to secure the IOL within the capsular bag and prevent IOL migration. Stability is therefore an important factor to avoid the need for surgery to reposition the lens. Each haptic includes a base adjacent to the optic, a distal foot portion and an intermediate portion connected between the base and the distal foot. The haptics can also be formed of a suitable biocompatible material, such as polymethacrylate, polypropylene and the like. While in some embodiments, the haptics can be formed integrally with the optic, in other embodiments, the haptics are formed separately and then attached to the optic. It should be appreciated that various haptic designs for maintaining lens stability and centration are known in the art, including, for example, C-loops, J-loops, and plate-shaped haptic designs. The present invention is readily employed with any of these haptic designs.

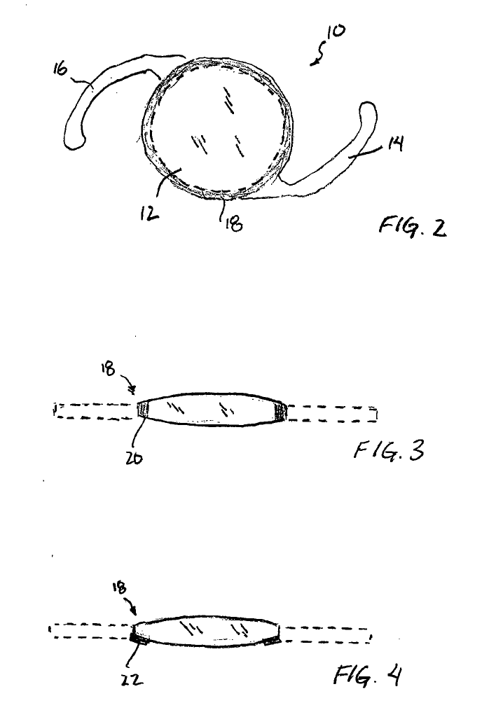

FIG. 3 shows one embodiment of the peripheral region 18 in which a portion of the optic is modified to render it opaque, e.g., by incorporation of a light absorbing dye. This opaque peripheral portion prevents internally-reflected peripheral rays from reaching the retina, e.g., by absorption. The term “opaque” as used herein, refers to an opacity that would result in a reduction in the intensity of the visible radiation, e.g., radiation with wavelengths in a range about 380 nm to about 780 nm, by more than about 25%, and preferably by more than about 50%, and most preferably by close to 100%. By way of example, in many embodiments, the intensity of the incident light passing through the opaque peripheral region is reduced by a factor greater than about 70 percent and more preferably greater than about 90 percent.

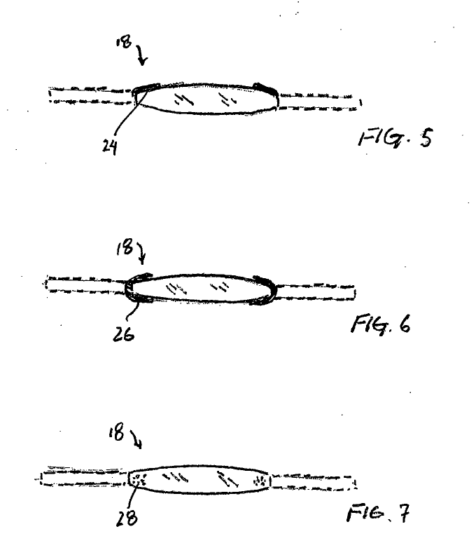

FIGS. 4-6 show other embodiments of the peripheral region 18 in which a portion of the optic is coated to render it opaque, e.g., by coating of a light absorbing dye. In FIG. 4, a coating 22 is applied to the posterior surface of the peripheral region. In FIG. 5, a coating 24 is applied to the anterior surface of the peripheral region. In FIG. 6, a coating 26 is applied to the entire edge including bother posterior and anterior surfaces of the peripheral region. In each case, the coatings serve to capture and redirect or absorb the high angle peripheral light rays that may enter eye and still miss a conventional optic.

FIG. 7 shows another embodiment of the peripheral region 18 in which a portion of the optic is modified to include light scattering material, either as a coating or by incorporation of scattering particles into the optic composition. The scattering material serves to diffuse internally reflected light and inhibit the formation of a secondary image or distinct visual artifact.

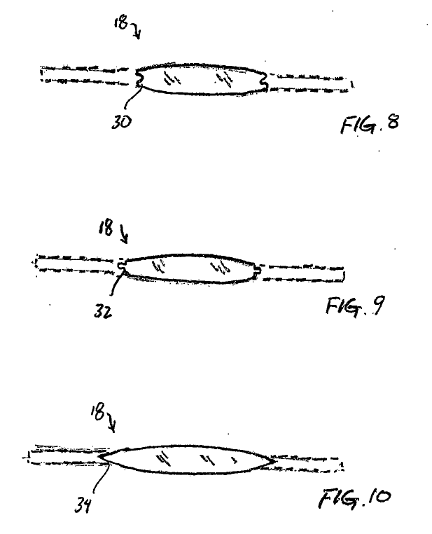

FIGS. 8-10 show other embodiments of the peripheral region 18 in which a portion of the optic edge is modified to inhibit transverse propagation of light. In FIG. 8, the edge of the optic includes a compound curved surface 30. In FIG. 9, another compound curve is shown, e.g., a radiused edge 32. The surface can also to textured in various ways to capture or redirect the light. In FIG. 10, a tapered edge 36 is shown (which can optionally include an opaque or light diffusing tip). Alternatively, the peripheral region can include a Fresnel lens for redirecting internally-reflected light to the retinal dark (shadow) region.

Additionally, one or both surfaces of the peripheral region can exhibit surface undulations (that is, the peripheral portion of the surface is textured) with amplitudes typically of the order of wavelengths of the visible light, e.g., less than about 1 micron, and preferably in the range of 0.2 microns to 0.4 microns. These surface undulations can cause scattering of peripheral light rays incident thereon, and hence inhibit formation of an image by those rays. Although some of the scattered rays might reach the retina, they do not lead to the formation of a strong secondary image that would result in perception of dark shadows. In fact, the scattering diverts a large portion of the incident peripheral rays to far peripheral portions of the retina that exhibit much reduced sensitivity.

The IOLs of the present invention can each be foldable, e.g., about an axis of their longer dimension to facilitate its implantation in the eye. More particularly, during cataract surgery, a clouded natural lens can be removed and replaced with the IOL 10. An incision is first made in the cornea to allow other instruments to enter the eye. The anterior lens capsule can be accessed via that incision to be cut in a circular fashion and removed from the eye. A probe can then be inserted through the corneal incision to break up the natural lens via ultrasound, and the lens fragments can be aspirated. An injector can be employed to place the IOL in a folded state in the original lens capsule. Upon insertion, the IOL can unfold and its haptics can anchor it within the capsular bag.

Once implanted in a patient's eye, the IOL can form an image of a field of view with the peripheral region receiving peripheral light rays entering the eye at large visual angles and either capturing the internally reflected rays or redirecting those rays (e.g., towards the principal image or by diffusion) to inhibit formation of a secondary image that could lead to perception of dark shadows. The term “large visual angles,” as used herein, refer to angles relative to the visual axis of the eye that are greater than about 50 degrees, and are typically in a range of about 50 degrees to about 80 degrees relative to eye's visual axis.

The central portion (or central optic) of the IOLs of the present invention can provide a single optical power or it can include a diffractive structure so as to provide multi-focal vision, e.g., both a far-focus optical power and a near-focus power. For example, the base curvature of the optic 12 can be selected such that the IOL provides a desired far-focus optical power, e.g., in a range of about −15 D to about 34 D. A diffractive structure disposed on the anterior surface provides a near focus optical power, e.g., in a range of about 1 D to about 4 D. The diffractive structure can include a plurality of diffractive zones (as known in the art) that are separated from one another by a plurality of steps.

In one embodiment, the diffractive zones are in the form of annular regions, where the radial location of a zone boundary (rᵢ) is defined in accordance with the following relation:

rᵢ²=(2i+1)λƒ Equation (1)

wherein
i denotes the zone number (i=0 denotes the central zone),
rᵢ denotes the radial location of the ith zone,
λ denotes the design wavelength, and
ƒ denotes an add power.

The steps can be uniform or they can exhibit a decreasing height as a function of increasing distance from the optical axis. In other words, the step heights at the boundaries of the diffractive zones are “apodized” so as to modify the fraction of optical energy diffracted into the near and far foci as a function of aperture size (e.g., as the aperture size increases, more of the light energy is diffracted into the far focus). Various apodization scaling functions can be employed, such as those disclosed in a co-pending patent application entitled “Apodized Aspheric Diffractive Lenses,” filed Dec. 1, 2004 and having a U.S. Ser. No. 11/000770 (Pub. No. 2006/0116764), which is herein incorporated by reference.

Those having ordinary skill in the art will appreciate that various changes can be made to the above embodiments without departing from the scope of the invention.

1. An intraocular lens (IOL), comprising
an optic with at least one peripheral region adapted to inhibit transverse propagation of internally reflected light rays.

2. The IOL of claim 1, wherein the IOL further comprises an optic body with a peripheral region that incorporates a light absorbing material.

3. The IOL of claim 1, wherein the IOL further comprises an opaque coating on at least a portion of a peripheral region of the optic.

4. The IOL of claim 1, wherein the IOL further comprises an opaque coating on at least a portion of an edge, a posterior surface or an anterior surface of the peripheral region of the optic.

5. The IOL of claim 1, wherein the IOL further comprises a light scattering composition on or within at least a portion of the peripheral region of the optic.

6. The IOL of claim 5, wherein the light scattering composition is a coating on at least a portion of the peripheral region of the optic.

7. The IOL of claim 5, wherein the light scattering composition is incorporated into at least a portion of the peripheral region of the optic.

8. The IOL of claim 1, wherein the IOL further comprises a compound curved surface on at least a portion of the peripheral region of the optic.

9. The IOL of claim 1, wherein the IOL further comprises a tapered edge on at least a portion the peripheral region of the optic.

10. The IOL of claim 9, wherein said tapered edge further comprises an opaque tip region.

11. The IOL of claim 9, wherein said tapered edge further comprises a light scattering tip region.

12. The IOL of claim 1, wherein said peripheral region comprises at least one refractive surface adapted to redirect said peripheral light rays.

13. The IOL of claim 1, wherein the optic comprises a diffractive structure to provide at least a far-focus optical power and a near-focus optical power.

14. The IOL of claim 1, wherein the optic comprises a transparent polymeric material.

15. The IOL of claim 1, wherein the optic comprises at least one polymeric material selected from the group of acrylics, acrylates, methacrylates, silicones, polypropylenes and hydrogels.

16. The IOL of claim 1, wherein the optic comprises a copolymer of acrylate and methacrylate materials.

17. The IOL of claim 1, wherein the optic comprises a cross-linked copolymer of 2-phenylethyl acrylate and 2-phenylethyl methacrylate.

18. A method of manufacturing an asymmetric intraocular lens (IOL), the method comprising forming an optic with at least one peripheral region adapted to inhibit transverse propagation of internally reflected light rays.

19. The method of claim 18, wherein the method further comprises joining the optic to at least one haptic.

20. The method of claim 18, wherein the method further comprises applying an opaque coating on at least a portion of a peripheral edge of the optic.

21. The method of claim 18, wherein the method further comprises applying an opaque coating on at least a portion of a posterior surface of the peripheral region of the optic.

22. The method of claim 18, wherein the method further comprises applying an opaque coating on at least a portion of anterior surface of the peripheral region of the optic.

23. The method of claim 18, wherein the method further comprises providing a light scattering composition on or within at least a portion of the peripheral region of the optic.

24. The method of claim 23, wherein the light scattering composition is a coating on at least a portion of the peripheral region of the optic.

25. The method of claim 23, wherein the light scattering composition is incorporated into at least a portion of the peripheral region of the optic.

26. The method of claim 18, wherein the method further comprises providing a compound curved surface on at least a portion of the peripheral region of the optic.

27. The method of claim 18, wherein the method further comprises providing a tapered edge on at least a portion of the peripheral region of the optic.

28. The method of claim 27, wherein the tapered edge further comprises an opaque tip region.

29. The method of claim 27, wherein the tapered edge further comprises light scattering tip region.

30. The method of claim 18, wherein the method further comprises providing at least one refractive surface adapted to redirect said peripheral light rays.

31. The method of claim 18, wherein the method further comprises providing a diffractive structure to provide at least a far-focus optical power and a near-focus optical power.

32. The method of claim 18, wherein the method further comprises selecting an optic that comprises a transparent polymeric material.

33. The method of claim 18, wherein the method further comprises selecting an optic that comprises at least one polymeric material selected from the group of acrylics, acrylates, methacrylates, silicones, polypropylenes and hydrogels.

34. The method of claim 18, wherein the method further comprises selecting an optic that comprises a copolymer of acrylate and methacrylate materials.

35. The method of claim 18, wherein the method further comprises selecting an optic that comprises a cross-linked copolymer of 2-phenylethyl acrylate and 2-phenylethyl methacrylate.

36. A method of reducing visual artifacts in an eye with an implanted intraocular lens (IOL), the method comprising:
providing an IOL having an optic with at least one peripheral region adapted to inhibit transverse propagation of internally reflected light rays,
implanting the IOL into an eye of a patient.

37. The method of claim 36, wherein the step of providing an IOL further comprises selecting an IOL with an opaque peripheral region.

38. The method of claim 37, wherein the opaque peripheral region further comprises a coating on at least a portion of the peripheral region of the optic.

39. The method of claim 37, wherein the opaque peripheral region further comprises a light absorbing composition is incorporated into at least a portion of the peripheral region of the optic.

40. The method of claim 36, wherein the step of providing an IOL further comprises selecting an IOL with a light scattering composition on or within at least a portion of the peripheral region of the optic.

41. The method of claim 40, wherein the light scattering composition further comprises a coating on at least a portion of the peripheral region of the optic.

42. The method of claim 40, wherein the light scattering composition further comprises a light scattering material incorporated into at least a portion of the peripheral region of the optic.

43. The method of claim 36, wherein the step of providing an IOL further comprises providing a compound curved surface on at least a portion of the peripheral region of the optic.

44. The method of claim 36, wherein the step of providing an IOL further comprises providing an IOL with a tapered edge on at least a portion of the peripheral region of the optic.

45. The method of claim 44, wherein said tapered edge further comprises an opaque tip region.

46. The method of claim 44, wherein said tapered edge further comprises light scattering tip region.

47. The method of claim 36, wherein the step of providing an IOL further comprises providing an IOL with a peripheral region that comprises at least one refractive surface adapted to redirect said peripheral light rays.

48. The method of claim 36, wherein the step of providing an IOL further comprises providing an IOL with an optic that comprises a diffractive structure to provide at least a far-focus optical power and a near-focus optical power.
